(19) United States
(12) Patent Application Publication
(10) Pub. No.: US 2025/0122088 A1
(43) Pub. Date: Apr. 17, 2025

(54) AMORPHOUS CALCIUM MAGNESIUM FLUORIDE PHOSPHATE PARTICLES

(71) Applicant: Psilox AB, Uppsala (SE)

(72) Inventor: Erik UNOSSON, Uppsala (SE)

(73) Assignee: Psilox AB, Uppsala (SE)

(21) Appl. No.: 18/834,254

(22) PCT Filed: Feb. 27, 2023
(86) PCT No.: PCT/SE2023/050171

§ 371 Date: Jul. 30, 2024

(30) Foreign Application Priority Data
Feb. 28, 2022 (SE) 2250275-1

(51) Int. Cl. C01F 11/22; A61K 6/20; A61K 33/42

(52) U.S. Cl. CPC C01F 11/22; A61K 6/20; A61K 33/42; C01P 2002/02; C01P 2002/90; C01P 2004/34; C01P 2004/64

(57) ABSTRACT

The present invention relates to X-ray amorphous and hollow calcium magnesium fluoride phosphate particles. The X-ray amorphous and hollow calcium magnesium fluoride phosphate particles have a mean diameter ranging from 50 to 500 nm and comprise a respective shell. The respective shell comprises 15-30 wt % calcium, 50-65 wt % phosphate, 4-8 wt % magnesium, 1-10 wt % fluoride, and the balance is water.

### FIELD OF THE INVENTION

The present invention relates to X-ray amorphous calcium magnesium fluoride phosphate particles, to methods of producing such particles and uses thereof.

### BACKGROUND

Dentin hypersensitivity is a clinical condition that can cause significant oral discomfort and pain, triggered by e.g., thermal, mechanical or evaporative stimuli. The underlying cause for the condition is that the dentin tubules have become exposed due to gingival recession or loss of enamel, in turn caused by e.g., abrasion or erosion, excessive tooth brushing or flossing, pocket reduction surgery or as a secondary reaction to periodontal disease.

Caries (tooth decay or dental cavities) is a major global healthcare issue and according to the 2015 Global Burden of Disease Study it is estimated that 2.3 billion people suffer from caries in their permanent teeth, and that 530 million children suffer from caries in primary teeth. According to the World Health Organization it is the most common non-communicable disease worldwide. Caries is a result of plaque formation on the tooth surface, where bacteria excrete acids while metabolizing fermentable carbohydrates. This causes demineralization of the enamel and dentin and, if allowed to progress, will cause formation of cavities. In order to prevent caries it is important to reduce the daily intake of dietary sugars and to exercise regular dental hygiene care, such as tooth brushing and flossing. Tooth brushing should be performed with a fluoridated toothpaste, which can prevent and arrest progression of caries by forming fluorapatite, i.e., inhibit demineralization.

Demineralization of dentin and enamel may be countered by remineralization, in which calcium and phosphate ions present in saliva or dentinal fluid can deposit to form new mineral. This requires a local supersaturation of ions and preferably a pH above neutral to form hydroxyapatite. In presence of fluoride, the newly formed mineral may be fluorapatite rather than hydroxyapatite, making it less soluble and more resistant to acid erosion. Since fluoride alone cannot substantially remineralize the tooth, it is pertinent that the saliva/dentinal fluid and the localized environment where remineralization needs to occur is supersaturated with calcium and phosphate ions. This is not always the case in natural saliva or dentinal fluid, and in caries-affected areas the calcium and phosphate ions may predominantly stem from demineralized enamel or dentin. As such there will still be a net mineral loss of the tooth structure. In order to regain mineral and tip the scale towards remineralization, additional calcium and phosphate ions are required.

Fluoride is recommended for the purpose of preventing dental caries, but excessive fluoride consumption or ingestion may lead to the development of dental fluorosis, particularly in young children during tooth formation. The condition can cause white spots or pitting of the enamel and has led to the recommendation to limit the level of fluoride in children's toothpaste and to control the fluoride levels in drinking water.

WO 2021/086252 discloses spherical and hollow calcium magnesium phosphate particles and compositions comprising such particles.

WO 2016/012452 A1 discloses a process for obtaining fluoride-doped citrate-coated amorphous calcium phosphate nanoparticles.

WO2011005896A1 discloses toothpaste droplets for delivering of toothpaste to a user.

There is still a need for calcium phosphate particles that could be used to prevent or treat at least some of the above-mentioned dental diseases or disorders. There is also a need to provide alternative fluoride sources that can limit or reduce the fluoride exposure while still providing the desired mineralization effect.

### SUMMARY

The present invention aims to solve the problems of the prior art by providing particles, a method of producing the particles, and a composition comprising the particles.

A first aspect of the invention relates to X-ray amorphous and hollow calcium magnesium fluoride phosphate particles having a mean diameter ranging from 50 to 500 nm. The X-ray amorphous and hollow calcium magnesium fluoride phosphate particles comprise a respective shell comprising:
15-30 wt % calcium of the total weight of the X-ray amorphous and hollow calcium magnesium fluoride phosphate particles;
50-65 wt % phosphate of the total weight of the X-ray amorphous and hollow calcium magnesium fluoride phosphate particles;
4-8 wt % magnesium of the total weight of the X-ray amorphous and hollow calcium magnesium fluoride phosphate particles;
1-10 wt % fluoride of the total weight of the X-ray amorphous and hollow calcium magnesium fluoride phosphate particles;
and wherein the balance is water.
   In one embodiment of the invention, the respective shell comprises:
18-30 wt % calcium, preferably 20-25 wt % calcium, of the total weight of the X-ray amorphous and hollow calcium magnesium fluoride phosphate particles;
50-60 wt % phosphate, preferably 52-57 wt % phosphate, of the total weight of the X-ray amorphous and hollow calcium magnesium fluoride phosphate particles;
5-8 wt % magnesium, preferably 6-7 wt % magnesium, of the total weight of the X-ray amorphous and hollow calcium magnesium fluoride phosphate particles;
3-10 wt % fluoride, preferably 3.2-5.8 wt % fluoride, of the total weight of the X-ray amorphous and hollow calcium magnesium fluoride phosphate particles;
and wherein the balance is water.

In one embodiment of the invention, the respective shell comprises:
20-25 wt % calcium of the total weight of the X-ray amorphous and hollow calcium magnesium fluoride phosphate particles;
52-57 wt % phosphate of the total weight of the X-ray amorphous and hollow calcium magnesium fluoride phosphate particles;
6-7 wt % magnesium of the total weight of the X-ray amorphous and hollow calcium magnesium fluoride phosphate particles;
3.2-5.8 wt % fluoride of the total weight of the X-ray amorphous and hollow calcium magnesium fluoride phosphate particles;
and wherein the balance is water.

In one embodiment of the invention, the respective shell comprises 3.2-10 wt % fluoride.

In one embodiment of the invention, the amount of calcium, magnesium and phosphorus in wt % is determined using ICP-OES, the phosphorous (P) is presumed to be present as phosphate (PO₄), and the amount of fluoride is measured using EDS or a fluoride ion selective electrode.

In one embodiment of the invention, the X-ray amorphous and hollow calcium magnesium fluoride phosphate particles are substantially spherical.

In one embodiment of the invention, the water is bound water.

Another aspect of the invention relates to a composition comprising a paste-forming compound and X-ray amorphous and hollow calcium magnesium fluoride phosphate particles according to the invention.

In one embodiment of the invention, the paste-forming compound is selected from the group consisting of glycerol, triglyceride, polyethylene glycol, polyvinyl alcohol, mineral oil, liquid paraffin, and any mixture thereof.

In one embodiment of the invention, the paste-forming compound is glycerol and the composition comprises 20-50 wt % of the X-ray amorphous and hollow calcium magnesium fluoride phosphate particles, based on the total weight of the composition.

A further aspect of the invention relates a method of manufacturing X-ray amorphous and hollow calcium magnesium fluoride phosphate particles. The method comprises the steps of:
formation of:
   a first salt solution comprising 10-200 mM Ca²⁺ and 5-120 mM Mg²⁺; and
   a second salt solution comprising 0-100 mM H₂PO₄⁻, 30-300 mM HPO₄²⁻, and 1-150 mM F⁻,
heating the first respectively the second salt solution separately to 60-90° C., forming a first, respectively second heated solution;
mixing the first heated solution with the second heated solution in a 1:1 volume ratio forming a mixed solution;
precipitation of particles from the mixed solution obtaining a slurry comprising particles; and
retrieving of the particles from the slurry.

In one embodiment of the invention, the method also comprises washing of the particles retrieved from the slurry.

Other aspects of the invention relate to X-ray amorphous and hollow calcium magnesium fluoride phosphate particles according to the invention or a composition according to the invention for use as a medicament, for use in prevention or treatment of caries, for use in prevention or treatment of dentin hypersensitivity or for use in prevention or treatment of white spot lesions.

Yet another aspect of the invention relates a dental product comprising a composition according to the invention. The dental product is selected from the group consisting of a toothpaste, a dentifrice, a dental varnish, a desensitizing gel, a mineralizing gel, a tooth whitening gel or strip, a dental prophy paste, a mouthwash, a tablet, a chewing gum, a dental sealant, a dental filling material, a dental cement, a dental pulp capping material.

In the following, the invention will be described in more detail, by way of example only, with regard to non-limiting embodiments thereof, reference being made to the accompanying drawings.

### LIST OF FIGURES

FIG. 1 a) and b) are scanning electron microscope (SEM) images of an embodiment of the invention;

FIG. 2 a) and b) are SEM images of another embodiment of the invention;

FIG. 3 is an X-ray diffraction (XRD) pattern of an embodiment of the invention;

FIG. 4 a), b) and c) are SEM images of embodiments of the invention;

FIG. 5 a) is a SEM image of a comparative example and b) is a SEM image of an embodiment of the invention;

FIG. 6 (top line, ACMFP) is an XRD pattern of an embodiment of the invention, and (bottom line, ACMP) is an XRD pattern according to a comparative example;

FIG. 7 is a graph from a fluoride release measurement according to an example of the invention;

FIG. 8 a) is a SEM image of an embodiment of the invention and b) is a SEM image of one comparative example;

FIG. 9 is an XRD pattern of an embodiment of the invention;

FIG. 10 a)-i) are SEM images of embodiments of the invention;

FIG. 11 is a graph from a fluoride release measurement according to an embodiment of the invention;

FIG. 12 a) is a SEM image of a comparative example, b) and c) are SEM images of embodiments according to the invention;

FIG. 13 (top, 40 mM F and middle, 10 mM F) are XRD patterns according to embodiments of the invention, and (bottom, 9 mM F) is an XRD pattern according to a comparative example;

FIG. 14 is a graph showing composition of particles according to an embodiment of the invention;

FIG. 15 is a graph from a fluoride release measurement according to an embodiment of the invention;

FIG. 16 a) is a SEM image according to one embodiment of the invention, and b) is an XRD pattern according to one embodiment of the invention; and

FIG. 17 is a flow-chart according to one aspect of the invention.

### DEFINITIONS AND ABBREVIATIONS

‘dentin hypersensitivity’—refers to dental pain arising from exposed dentin surfaces on teeth in response to a stimuli, e.g., thermal stimuli, may also be referred to as ‘sensitive teeth’;

‘mean particle size’—refers to the mean or average particle size of individual particles and fused or aggregated particles forming small clusters. Mean particle size is determined using dynamic light scattering (DLS), or scanning electron microscopy (SEM);

‘bound water’—refers to water of hydration associated with the amorphous calcium phosphate particles according to the invention, based on the generalized chemical formula of amorphous calcium phosphate as CaₓH_{y}(PO₄)_{z}·nH₂O, n=3-4.5.

‘X-ray amorphous’—refers to a material or particles that lacks long range crystalline order. The crystallinity or X-ray or XRD amorphous state of the particles is determined by powder X-ray diffraction. (XRD) using Cu-Kα (1=1.5406 Å), scanning 2θ from 7 to 60° with a step size of 0.0125°. A crystalline material reflects the X-rays according to the arrangement of its crystallographic planes and generates an identifiable pattern of sharp peaks, whereas an X-ray amorphous material only generates a single broad diffuse peak. Herein, the particles are, thus, classified as X-ray amorphous if the generated pattern lacks identifiable sharp peaks and is only characterized by a broad diffuse peak;

‘ACMFP’—is short for amorphous calcium magnesium fluoride phosphate;

‘ACMP’—is short for amorphous calcium magnesium phosphate;

‘ACP’—is short for amorphous calcium phosphate; and

‘wt %’—refers to weight percent of the ingredient in relation to the total weight of the particles or the composition.

### DETAILED DESCRIPTION

Different calcium phosphate technologies and particles have been introduced in oral care products to promote remineralization of enamel as well as occlusion of dentin tubules for reduction of dentin hypersensitivity. One of the most effective approaches is to use amorphous calcium phosphate (ACP), which, with its high aqueous solubility, can supply a high concentration of calcium and phosphate ions, making it highly bioactive. The metastable nature of ACP also means that it is easily transformed into more stable calcium phosphate phases, such as hydroxyapatite. In fact, ACP is considered a precursor of natural apatite in teeth and therefore plays an important role in endogenous mineralization.

A challenge in formulating oral care products, such as toothpastes or dentifrices, with ACP is reactivity with fluoride. Part of the effectiveness of fluoride to inhibit caries and demineralization is its reactivity and resemblance to hydroxide ions (OH⁻), which it substitutes for in hydroxyapatite to form fluorapatite. It can also react with calcium and magnesium ions to form poorly soluble salts, such as CaF₂ and MgF₂. Stabilized compositions containing both ACP particles and fluoride enable more effective treatment of both caries and dentin hypersensitivity.

The present invention provides particles of amorphous calcium magnesium fluoride phosphate (ACMFP) as well as a method for producing the particles. The particles are stable upon storage, in the sense that they do not crystallize but remain X-ray amorphous. The as-synthesized particles are stable during storage for at least a time period of a month. The particles in composition with a paste-forming compound are stable for at least a time period of a year. The particles may be applied in oral care products, such as toothpastes, dentifrices, fluoride varnish or desensitizing/mineralizing gels to prevent caries and/or prevent or treat dentin hypersensitivity.

A first aspect of the invention relates to X-ray amorphous and hollow calcium magnesium fluoride phosphate particles having a mean diameter ranging from 50 to 500 nm. The X-ray amorphous and hollow calcium magnesium fluoride phosphate particles comprise a respective shell comprising:
15-30 wt % calcium;
50-65 wt % phosphate;
4-8 wt % magnesium;
1-10 wt % fluoride; and wherein the balance is water. All the wt % are given as of the total weight of the X-ray amorphous and hollow calcium magnesium fluoride phosphate particles.

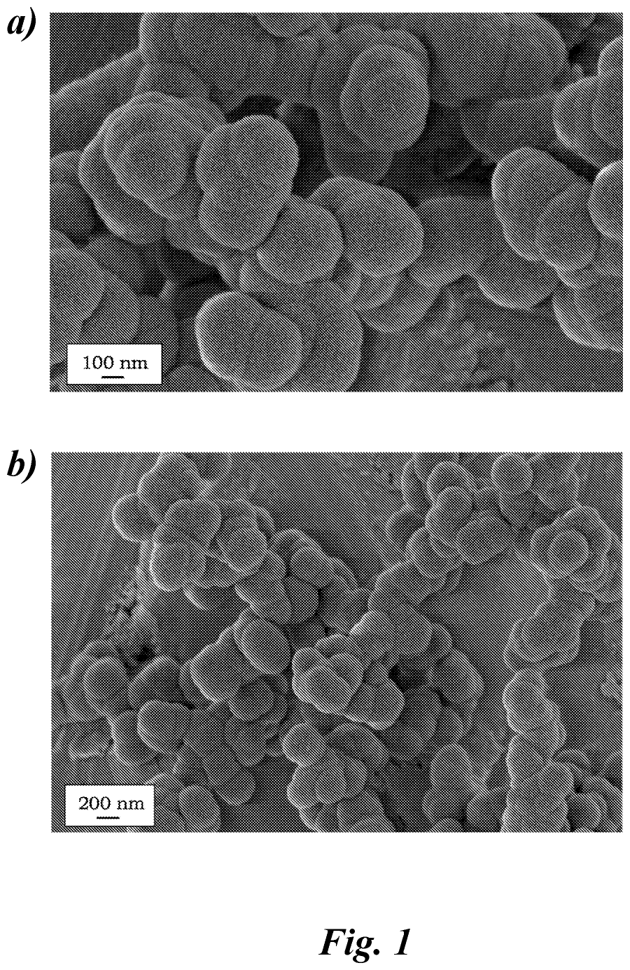

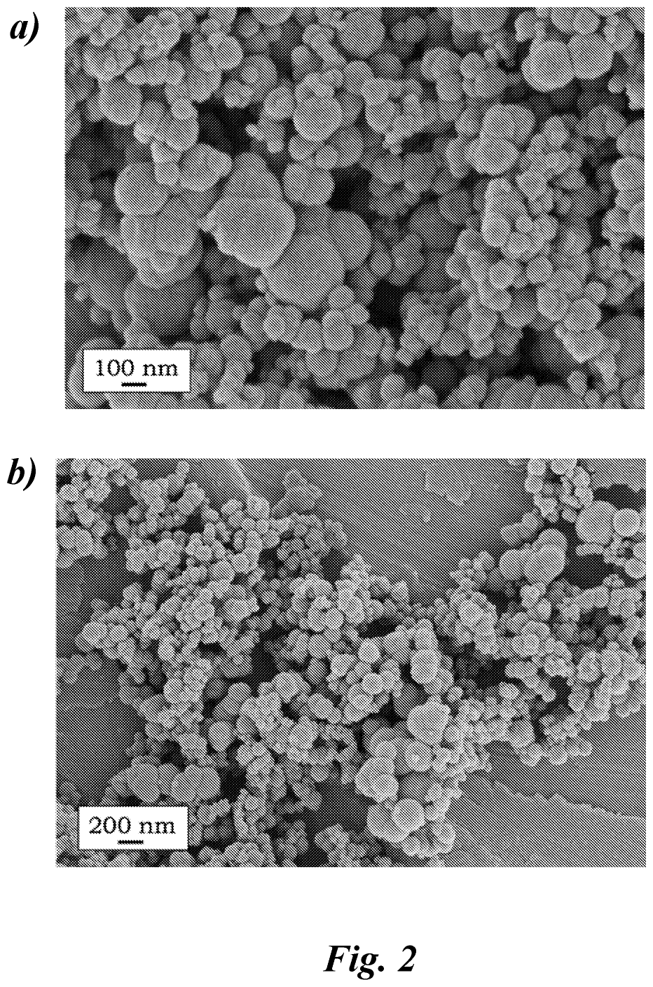

In one embodiment of the invention, the particles are predominantly spherical in shape, comprising a hollow core surrounded by an outer shell. Individual particle size ranges from 50 to 500 nm in diameter, or 100-300 nm in diameter, whereas clusters of fused or aggregated particles may be several μm. FIGS. 1a and 1b and FIGS. 2a and 2b show SEM images of particles according to embodiments of the invention. The particles are well suited in size and shape to penetrate exposed dentin tubules, where rapid conversion into fluorapatite can occur to occlude the tubules with a well adhered and acid resistant mineral that will restrict fluid motions and thereby provide hypersensitivity relief according to the predominant hydrodynamic theory. In one embodiment, the particles are substantially spherical.

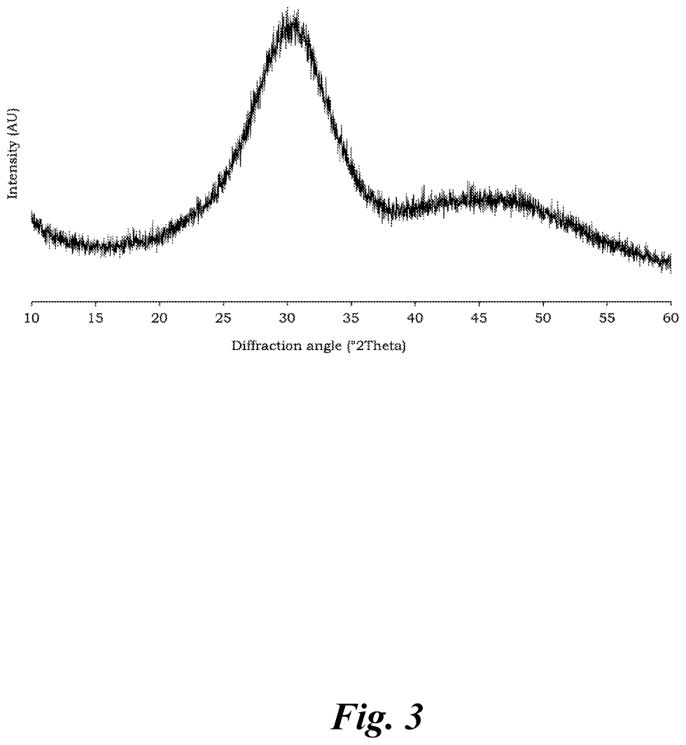

The X-ray amorphous and hollow calcium magnesium fluoride phosphate particles of the present invention are core-shell particles where the core is hollow and the shell of the particles is composed of 15-30 wt % calcium, 50-65 wt % phosphate, 4-8 wt % magnesium, 1-10 wt % fluoride, and wherein the balance is water. The general appearance of the particles is shown in the SEM images in FIGS. 1 and 2. The particles are X-ray amorphous, as demonstrated in FIG. 3. Without being bound by theory, the amorphous character of the particles is believed to be caused by magnesium ion substitution and the conditions during particle precipitation. The inclusion of fluoride (F⁻) in the particles is, without being bound by theory, believed to be caused by replacement of hydroxyl groups (OH⁻), otherwise likely to be present on or near the surface of the particles to counter positive charges of calcium and magnesium. In one embodiment, the water refers to ‘bound water’, i.e., water that is part of the chemical composition.

A considerable challenge in utilizing ACP in biomedical applications, such as dentin and enamel mineralization and tubule occlusion, is to stabilize it in production and product formulations. Aqueous formulations with ACP and long-term storage of ACP formulations tend to crystallize the material, making it less bioactive. A way of stabilizing ACP is to substitute part of the calcium with magnesium to form ACMP, which, with its smaller ionic radius, will disrupt the active crystallite growth sites.

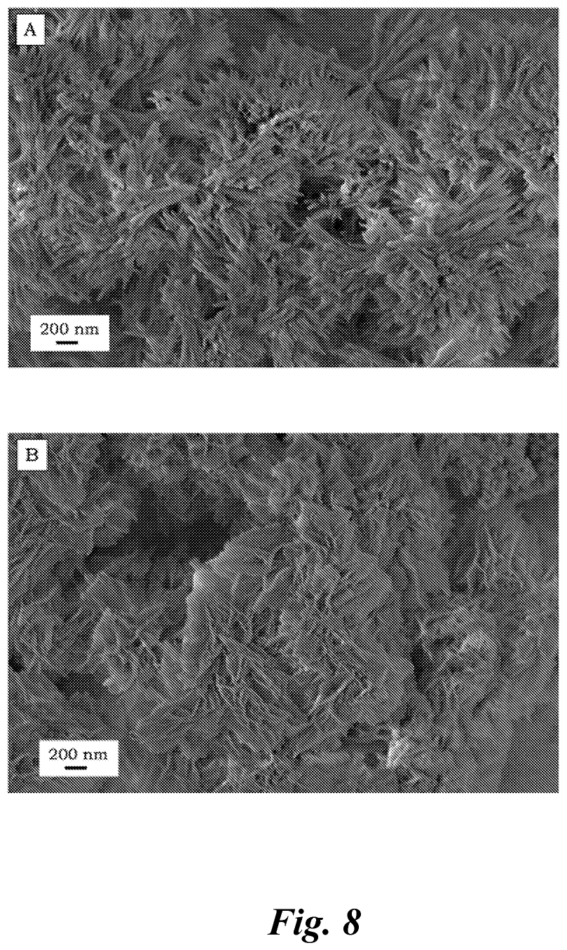

The principal and functional difference between ACMP and ACMFP particles lies in their composition and crystallization product in oral conditions, where ACMP particles will form poorly crystalline hydroxyapatite whereas ACMFP particles will form sharp crystals of fluorapatite, as demonstrated in FIG. 8 and Example 3. The conditions necessary to synthesize the ACMFP particles are not obvious for someone skilled in the art even with the background provided for ACMP particle synthesis in WO 2021/086252, as fluoride in combination with high concentrations of calcium ions will generally precipitate as poorly soluble CaF₂. In order to avoid CaF₂ formation and retain the amorphous and spherical character of the particles, as well as the long-term stability to enable oral product formulation, the fluoride addition must be deliberate and according to the method described in the present invention.

It is a significant advantage with the invention that the deliberate incorporation of fluoride in the particles does not lead to immediate crystallization of the particles, and that they do not primarily form CaF₂ or other poorly soluble salts. It is another significant advantage with the invention that particles according to the invention remain stable over time to allow product formulation and effective tooth mineralization.

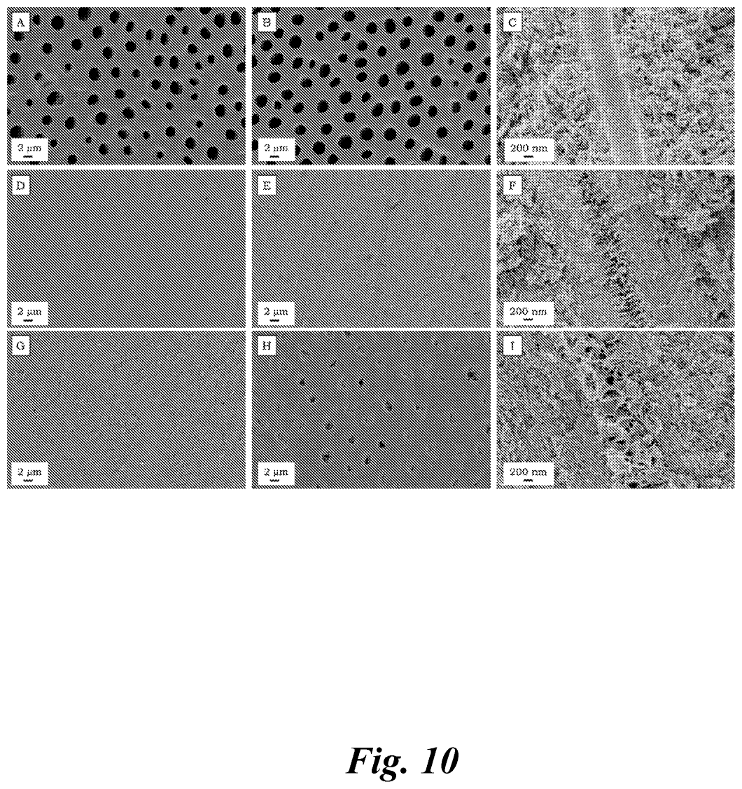

It is a further advantage with the invention that the composition and amorphous nature of the particles may enable efficient mineralization of dentin and enamel by providing the required ions and nucleation sites. One advantage over ACP or ACMP is that the ACMFP particles according to the invention have the ability to form fluorapatite directly without the need or use of any additional fluoride source. The advantage of ACMFP over ACMP in forming an acid resistant mineralization layer is demonstrated in FIG. 10, where images in FIGS. 10E and 10H show the appearance of dentin after treatment with ACMFP and ACMP particles, respectively, and an acid challenge. The ACMFP-treated tubules are still completely occluded whereas the ACMP-treated tubules have reappeared.

The presence of fluoride in the ACMFP particles does not change the general appearance/morphology or the crystalline state of the particles, and the degree of fluoride doping or inclusion can be adjusted in the particle production process described in the invention. Whereas the potential inclusion of fluoride or other ions, such as sodium, potassium, silicon/silicate, or zinc in ACMP particles described in WO 2021/086252 may be viewed as impurities (<1 wt %), the fluoride inclusion in ACMFP particles of the present invention is deliberate and purposeful with the intention to increase the mineralization and caries prevention potential of the particles in oral care products. With the invention herein it is realized and demonstrated that fluoride not only can be impurities but added to the particles in concentrations that are clinically relevant. Furthermore, the disclosure in WO 2021/086252 actually does not include any examples or enabled embodiments of ACMP particles comprising fluoride.

Incorporation of fluoride in the ACMFP particles, thus, enables introduction of a fluoride treatment that is not only able to arrest or prevent tooth demineralization, but actively promote remineralization and reverse the local progression or caries. However, the fluoride incorporation is not trivial. Mixing solutions of NaF and CaCl₂ generally results in the immediate precipitation of CaF₂ particles. In one such example with concentrations similar to the present invention (8 mM NaF and 40 mM CaCl₂), cubic and solid CaF₂ particles with sides of 2-3 μm were formed. Hence, to obtain pure ACMFP particles without simultaneous precipitation of CaF₂, the method described herein can be followed, which also allows for fluoride inclusion of up to 10 wt %.

It is an advantage with the invention that ACMFP particles comprising fluoride, which upon delivery to demineralized enamel or exposed dentin, can provide an opportunity for remineralization to occur, as particle transformation combined with dissolution and reprecipitation can result in a net mineral gain. It is a further advantage with the invention that ACMFP particles comprising fluoride can be used as a fluoride source in, for example, a toothpaste. This can reduce the amount of added fluoride since less ion excess is needed due to a localized supersaturation on the tooth surface, which is beneficial for the environment and the health of the user.

The fluoride concentration in oral care products varies depending on the type of product and frequency of application. Conventional fluoride toothpaste contains 1,000-1,500 ppm F and has been proven clinically effective in controlling caries in a wide range of studies. Higher fluoride content toothpaste (5,000 ppm F) is generally prescribed to at-risk patients. Fluoride varnish typically contains 22,500 ppm F and may be applied topically by the dentist at routine visits. A conventional toothpaste with 1,500 ppm F could, thus, be formulated with 5 wt % ACMFP particles according to the invention containing 3 wt % F. A 5,000 ppm F toothpaste could further be formulated with 10 wt % ACMFP containing 5 wt % F, or reversely with 5 wt % ACMFP containing 10 wt % F.

Herein, the term ‘mineralization’ refers partly to the process, in which calcium, phosphate, and sometimes fluoride ions deposit to form hydroxyapatite-like mineral of various degree of crystallinity from a supersaturated solution. In cases where fluoride is present, the mineral formed may be fluoride substituted hydroxyapatite or fluorapatite. The amorphous calcium magnesium fluoride phosphate particles according to the invention may also undergo a direct phase transformation, or crystallization process, into fluoride substituted hydroxyapatite. Both these processes (i.e., deposition of ions from a supersaturated solution and direct phase transformation/crystallization) can occur simultaneously or be combinatorial in an aqueous environment, such as saliva or dentinal fluid. For the purpose of clarity, the term ‘mineralization’ is used with the intention to encompass both of the above-mentioned processes.

The particles of the present invention are composed of amorphous calcium magnesium fluoride phosphate, and the particles are arranged to provide the calcium, phosphate, and fluoride ions required to form and maintain an acid-resistant mineralization layer on the tooth surface, all from a same particle source. Hence, it is an advantage with the invention that during use of the particles there is no need for another component in order to form and maintain an acid resistant mineralization layer on the tooth surface. The mineralization effect of the particles can prevent dental caries and reduce hypersensitivity by effectively mineralizing enamel and occluding exposed dentin tubules. With the addition of fluoride to the amorphous calcium magnesium phosphate particles, the present inventor has demonstrated that a crystalline layer of fluorapatite rapidly forms on the tooth surface as well as within the dentin tubules without the need of any other fluoride source, such as NaF.

One of the advantages with particles according to the invention is that the ions required to form a mineralization layer of fluorapatite are all present from the same source, meaning that as long as the particles are delivered to the enamel or dentin, they will act to mineralize the area directly, in part by creating a local supersaturation of ions. By contrast, other mineralization agents claiming to deliver calcium, phosphate, and fluoride ions, predominantly by dissolution of different salts or minerals, must rely on separate dissolution, large scale ion diffusion, and a certain degree of probability to ensure that sufficient amounts of the correct ions are present to result in local supersaturation and subsequent precipitation of hydroxyapatite and/or fluorapatite at the point of treatment.

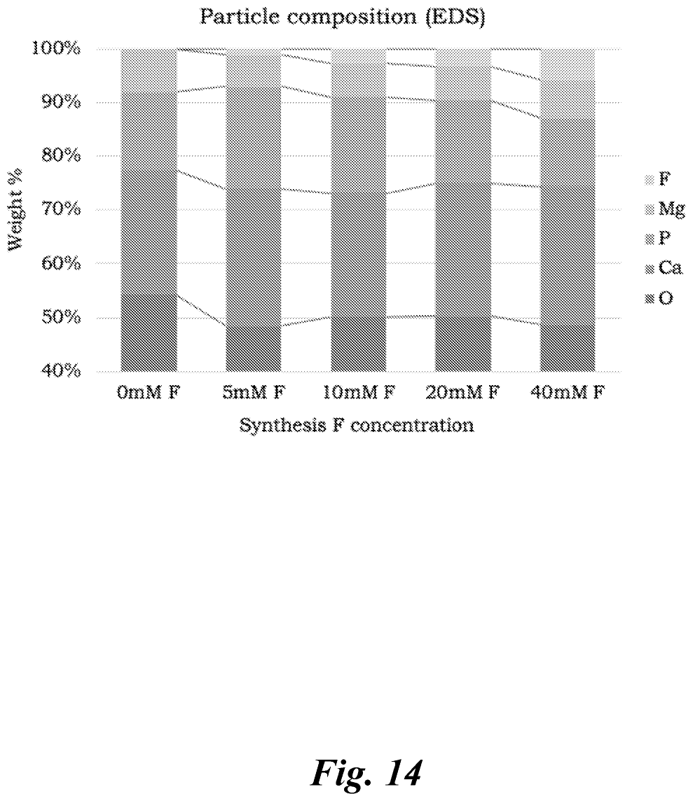

FIG. 14 and Table 1 below show the elemental composition of different examples of the invention, synthesized to comprise varied amounts of fluoride. Sample 1 was synthesized without fluoride to serve as a reference. The composition in FIG. 14 and Table 1 was determined by energy-dispersive X-ray spectroscopy (EDS), using an acceleration voltage of 6 kV and a working distance of 8 mm. All samples in Table 1 formulated with fluoride have the ability to form an acid resistant mineralization layer as described above.

TABLE 1
Elemental composition (wt %) of different examples according
to the invention.

| Element | Sample 1 | Sample 2 | Sample 3 | Sample 4 | Sample 5 |
|---|---|---|---|---|---|
| O | 54.2% | 48.4% | 50.1% | 50.3% | 48.6% |
| Ca | 23.2% | 25.5% | 23.0% | 24.7% | 25.7% |
| P | 14.6% | 19.0% | 17.9% | 15.5% | 12.8% |
| Mg | 7.9% | 5.9% | 6.4% | 6.3% | 7.1% |
| F | 0.1% | 1.2% | 2.6% | 3.2% | 5.8% |

Determination of the chemical composition of the particles of the invention can be done by e.g., EDS as demonstrated in FIG. 14 and Table 1. Such analysis is generally performed under vacuum in a scanning electron microscope (SEM) equipped with an EDS detector, which works by quantifying characteristic X-rays emitted from elements in the sample. Benefits of this analysis are that all elements of interest (except hydrogen) may be detected and quantified, as listed in Table 1, and the composition of individual particles may be determined. Depending on the sample preparation, however, some additional elements not pertaining to the particles may also be detected, such as Al, Au, Pd, C etc., coming from the sample substrate or the sputtering process to make the particles electrically conductive for analysis. These elements should be selectively removed to obtain a viable composition. The compositional result is also compiled to 100% with the selected elements, meaning that there is a risk of over- or underestimating certain elements if e.g., there is an abundance of additional elements or if the particles become dehydrated from their original state due to the vacuum drying. Complementary methods for determining the particle composition or specific features are, thus, warranted, such as inductively coupled plasma-optical emission spectroscopy (ICP-OES). With ICP-OES very precise measurements can be performed, and the method quantifies the elemental contents of a dissolved sample rather than specific particles. The drawback with the method, however, is that fluoride is not detectable. A typical method for determining fluoride in a sample is to use a fluoride ion selective electrode, which may complement ICP-OES measurement and is also beneficial for measuring e.g., fluoride release from a sample or material. Obtaining a complete and reliable composition of the particles of the invention may, thus, require the use of multiple analysis techniques, and results should be considered in light of the limitations of each method. In one embodiment, the method for quantifying the fluoride concentration in the particles is by EDS as described above. In one embodiment, the method for determining Ca, Mg, and P concentrations in the particles is by ICP-OES. In one embodiment, the phosphorous is presumed to be present as phosphate (PO₄). In one embodiment, the method for determining fluoride release from the particles is by using a fluoride ion selective electrode or EDS.

In one embodiment of the invention, the respective shell of the X-ray amorphous and hollow calcium magnesium fluoride phosphate particles comprises:
18-30 wt % calcium, preferably 20-25 wt % calcium;
50-60 wt % phosphate, preferably 52-57 wt % phosphate;
5-8 wt % magnesium, preferably 6-7 wt % magnesium;
1-10 wt % fluoride, preferably 3.2-5.8 wt % fluoride or 3-5 wt % fluoride, and wherein the balance is water.

In a particular embodiment of the invention, the respective shell of the X-ray amorphous and hollow calcium magnesium fluoride phosphate particles comprises:
20-25 wt % calcium;
52-57 wt % phosphate;
6-7 wt % magnesium;
3.2-5.8 wt % fluoride, and wherein the balance is water.

In one embodiment of the invention, the respective shell of the X-ray amorphous and hollow calcium magnesium fluoride phosphate particles comprises 3.2-10 wt % fluoride.

In one embodiment of the invention, the water is bound water.

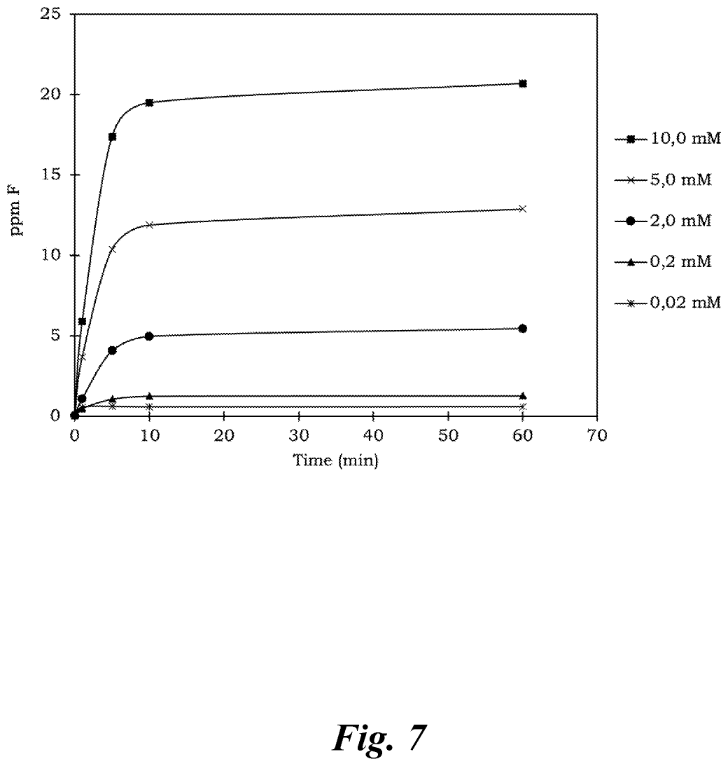

FIG. 7 shows a graph from a release measurement. Fluoride release was measured from amorphous calcium magnesium fluoride phosphate particles according to the present invention. The release was measured using an ion selective electrode in total ionic strength adjustment buffer (TISAB) II solution with a 0.1% by weight particle addition. As can be seen >90% of the total fluoride measured after 60 minutes was released within the first 10 minutes for all samples. Approximately 80% was released within 5 minutes. It is an advantage with the invention that the particles can release most of the incorporated fluoride rapidly.

A second aspect of the invention relates to a composition comprising a paste-forming compound and X-ray amorphous and hollow calcium magnesium fluoride phosphate according to the invention.

To obtain a stable composition, particles according to the invention are mixed with a paste-forming compound, for instance glycerol. In one embodiment, the paste-forming compound is essentially water-free, such as <10 wt % water, preferably <5 wt % water and more preferably <1 wt % water.

It is an advantage with a composition according to the invention that the particles can be homogenously dispersed in the composition. It is further advantageous that the composition has good flowability and/or viscosity and can easily be mixed with other ingredients to prepare, for example, a toothpaste.

Glycerol is a preferred paste-forming compound since it is widely used and generally accepted as an ingredient in pharmaceutical, cosmetic, and personal care products. It is easily soluble in water and has a high boiling point. In a process for making the composition according to the invention glycerol may be selectively retained in the composition while excess free water is evaporated. Evaporating at least most of the excess water can be beneficial in terms of shelf-life of the composition.

In one embodiment of the invention, the paste-forming compound is selected from the group consisting of glycerol, triglyceride, polyethylene glycol, propylene glycol, polypropylene glycol, polyvinyl alcohol, mineral oil, liquid paraffin, and any mixture thereof.

Even while stable for some time periods, such as a few months, particles according to the invention tends to crystallize into more stable forms of calcium phosphate upon long term storage, i.e., several months. A composition comprising particles according to the invention and a paste-forming compound can enable long-term stability of the particles, i.e., several years.

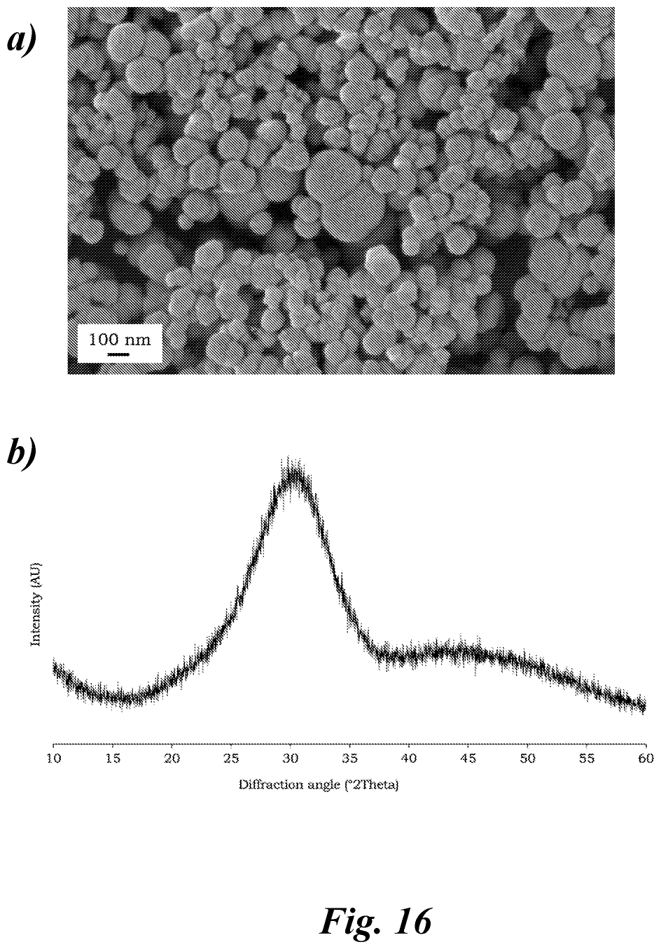

Such a composition according to the second aspect of the invention can be manufactured by mixing particles according to the invention with a paste-forming compound, for instance glycerol, and dry the mixture to remove any excess water. Drying may take place in e.g., a vacuum oven or a forced convection oven at 60-90° C., or in a combined dryer/mixer equipment. The dried composition is then preferably homogenized using a suitable mixer to obtain a paste, which should contain <10% excess water to maintain long term stability of the particles. Forming a homogeneous and stabilized paste enables good dispersion of the particles during storage and facilitates easier mixing with other ingredients to formulate e.g., a toothpaste, a desensitizing gel, a mineralizing gel, or other dental products. A composition with desired properties comprises 20-50 wt % particles according to the invention and 50-80 wt % glycerol. FIG. 16 demonstrates the stability of the particles in such a composition after three months storage at 40° C., generally representative of at least one year storage or more at room temperature. FIG. 16a shows a SEM image of such particles from which it is clear that the particles have maintained their spherical shape, shell morphology and their size. FIG. 16b shows a XRD diffractogram of such particles from which it is clear that the particles are still X-ray amorphous.

In one embodiment of the invention, the paste-forming compound is glycerol and the composition comprises 20-50 wt % of the X-ray amorphous and hollow calcium magnesium fluoride phosphate particles, based on the total weight of the composition.

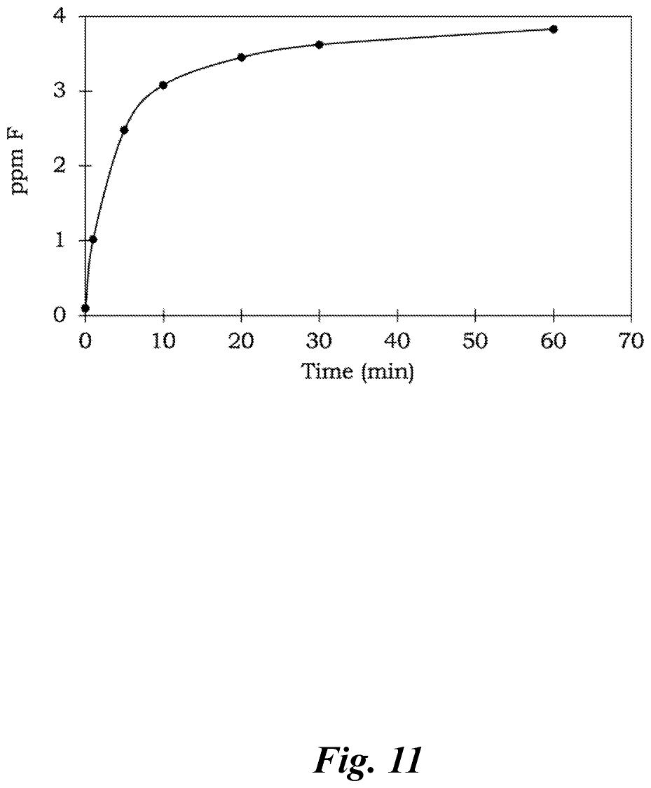

FIG. 11 shows a release measurement from a composition according to the invention. Fluoride release was measured from a paste comprising glycerol and amorphous calcium magnesium fluoride phosphate particles according to the present invention. The release was measured using a fluoride ion selective electrode with 1 mg/mL paste in TISAB II. As can be seen most of the fluoride was released during the first 10 minutes. It is an advantage with the invention that the composition can release most of the incorporated fluoride rapidly.

Particles according to the invention can also be delivered as a slurry, i.e., in the form of a mixture of a solvent, or mixture of solvents, and particles. The solvent can, for example, be ethyl alcohol, isopropyl alcohol, water, or a mixture of those.

A third aspect of the invention relates to a method of manufacturing X-ray amorphous and hollow calcium magnesium fluoride phosphate particles (ACMFP particles), which may be performed in either a single fluidic solution process or in a process where two separate fluidic solutions are mixed.

In the single solution process salts are dissolved one by one at or around room temperature (15-25° C.) to form a clear solution comprising 100-200 mM NaCl, 0.5-2.0 mM CaCl₂, 0.3-1.2 mM MgCl₂, 0.5-2.0 mM KH₂PO₄, 4.0-16.0 mM Na₂HPO₄, and 0.1-10.0 mM NaF. The pH of the solution should be 6.5-9.5 and may be adjusted by altering the ratio of H₂PO₄⁻/HPO₄²⁻ ions. One skilled in the art understands that the salts mentioned may be exchanged to encompass also other counter ions than Na⁺, Cl⁻, and K⁺, and that the final solution may also be formed from pre-dissolved stock solutions. What is not disclosed in the prior art and also not obvious to someone skilled in the art is, among other things, the degree, to which fluoride may be added to the solution to seamlessly increase the fluoride concentration in the resulting particles, whilst still maintaining the morphological properties and amorphous character of the particles, as demonstrated in e.g., Example 1. In order to formulate a final product such as a toothpaste with clinically effective fluoride concentration (1,000-1,500 ppm F), the particles should contain at least 1 wt % F, preferably 3-5 wt % F, or 3.2-5.8 wt % F, to facilitate toothpaste formulation and minimize added costs. In order to obtain a clear solution, from which ACMFP particles according to the invention can be precipitated using the single solution process, the salts should be dissolved in the order listed above to form a stable buffer solution, or else other particles are likely to precipitate directly. The H₂PO₄⁻/HPO₄²⁻ ions should preferably be added carefully under agitation as a pre-dissolved stock solution, and the fluoride ions should be added last as a pre-dissolved stock solution, titrating with care under stirring to avoid any premature precipitation. The formed solution is then heated under stirring at a rate exceeding 1° C./min to a temperature of 60-90° C., at which point precipitation of ACMFP particles will occur. One skilled in the art understands that the starting temperature and the end temperature is not required to be precisely defined; but as disclosed herein the key is to start with a clear solution, from which particles can precipitate, and the rate of precipitation will then essentially be determined by the end temperature according to the laws of thermodynamics. Room temperature is practical from a salt mixing perspective, and the elevated 60-90° C. in order to get a temperature below the boiling temperature of the salt solutions (100° C.). Retrieval of particles may be performed by e.g., filtration immediately or after a holding time at 60-90° C. for up to 1 hour. Retrieved particles are then optionally, but preferably washed with e.g., water or alcohol to remove salt residues.

In the two-solution process, two separate salt solutions are prepared at or around room temperature (15-25° C.); one containing 10-200 mM CaCl₂ and 5-120 mM MgCl₂; and one containing 0-100 mM KH₂PO₄, 30-300 mM Na₂HPO₄, and 1-150 mM NaF. One skilled in the art understands that also these salt solutions may be prepared using other counter ions than Na⁺, Cl⁻, and K⁺. What is not obvious to someone skilled in the art and also not disclosed in the prior art is the importance of adding the fluoride ions to the phosphate solution, i.e., the solution containing the H₂PO₄⁻ and/or HPO₄²⁻ ions. Adding small amounts (<1 mM) of fluoride to the Ca²⁺ and Mg²⁺ solution may result in trace level inclusion (<1 wt %) of fluoride in the resulting particles, but any further addition (>1 mM) will result in CaF₂ precipitation, effectively prohibiting the formation of ACMFP particles. As such, ACMFP particles with any significant amount of fluoride inclusion (>1 wt %) can only be synthesized in the two-solution process of the invention if the fluoride ions are added to the phosphate starting solution. The two solutions are then heated separately to 60-90° C. before being mixed at a 1:1 volume ratio, which may be performed by adding the two solutions at a similar rate to a common container, or by mixing the two solutions in a continuous flow process. The particles will precipitate immediately upon mixing the two solutions. The formed particles are then retrieved by e.g., filtration or centrifugation, and washed with e.g., water or alcohol to remove counter ions and salt residues.

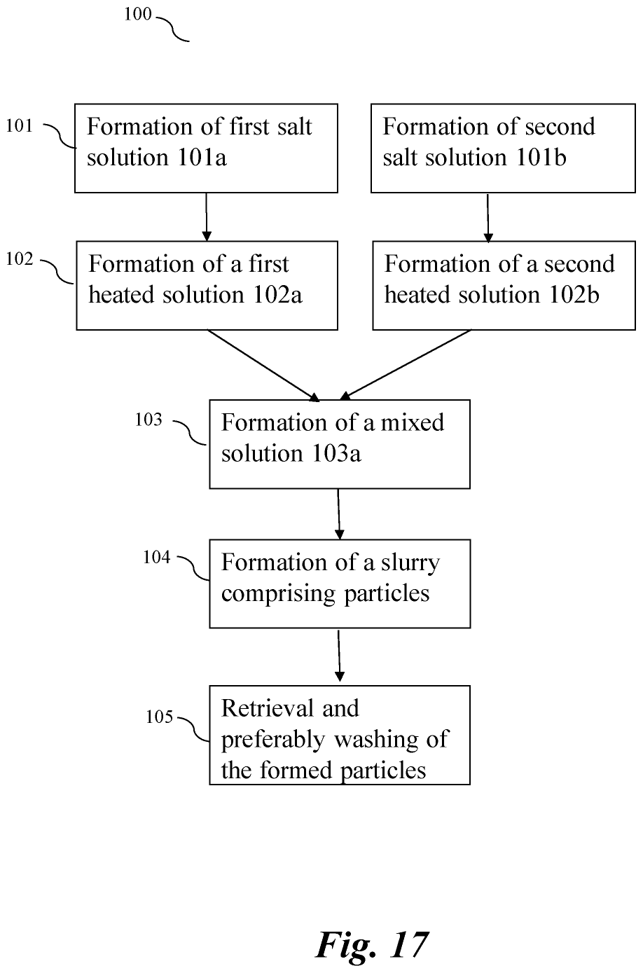

In other words, a method 100 for manufacturing X-ray amorphous and hollow calcium magnesium fluoride phosphate particles according to the invention, see FIG. 17, comprises the steps of:
formation 101 of:
   a first salt solution 101a comprising 10-200 mM Ca²⁺, such as CaCl₂, and 5-120 mM Mg²⁺, such as MgCl₂; and
   a second salt solution 101b comprising 0-100 mM H₂PO₄⁻, such as KH₂PO₄, 30-300 mM HPO₄²⁻, such as Na₂HPO₄, and 1-150 mM F⁻, such as NaF,
heating 102 the first 101a respectively the second 101b salt solution separately to 60-90° C., forming a first 102a, respectively second 102b heated solution;
mixing 103 the first heated solution 102a with the second heated solution 102b in a 1:1 volume ratio forming a mixed solution 103a;
precipitation 104 of particles from the mixed solution 103a obtaining a slurry comprising particles; and
retrieving 105 of the particles from the slurry.

The method 100 may optionally, but preferably, also comprise washing of the particles retrieved from the slurry.

The method for synthesizing the particles according to the present invention may be both replicable and scalable, and the particles may be stabilized by forming a composition with a paste-forming compound, such as glycerol.

It is an advantage with the method that by preparing and heating the first 101a and second 101b salt solution separately the concentrations of the respective salt solution can be higher and, thus, less excess water and energy is needed. It is a further advantage with the method that by preparing the first 101a and second 101b solutions separately, the primary cations (Ca²⁺ and Mg²⁺) and anions (H₂PO₄⁻, HPO₄²⁻ and F⁻) are separated and no added counter ions (i.e., NaCl) are required to stabilize the solutions. This is beneficial in terms of natural resources, time and energy consumption since less excess salt is required to form the particles, and as a result less excess salt has to be disposed of or recuperated.

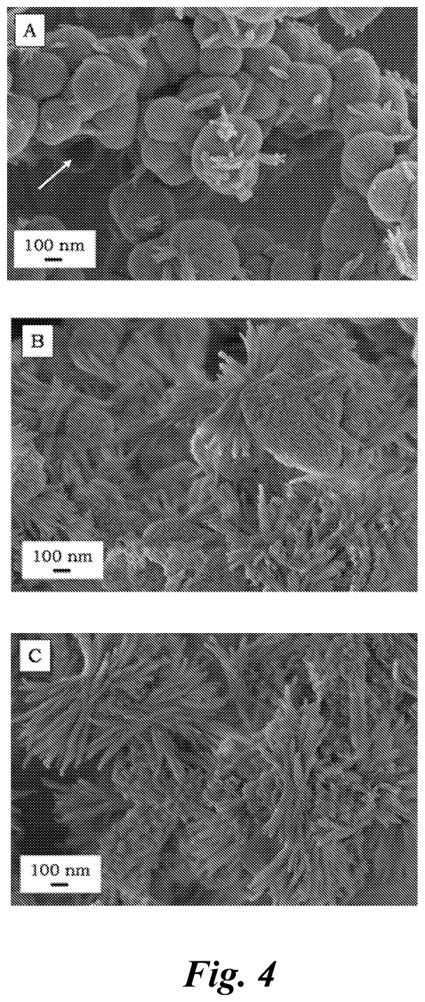

The degree of fluoride inclusion in the particles of the present invention is adjustable by controlling the concentration of fluoride ions in relation to other ions in the starting solutions. Increasing the amount of fluoride while maintaining the same amount of other relevant ions results in an increased level of fluoride inclusion in the particles, all while other characteristics such as appearance and crystallinity remain unchanged, see Examples 2 and 5. As to the present inventor's best knowledge, this feature is unique and not disclosed in any prior art. The fluoride ion release from the particles is adjustable accordingly, with both the total amount and the rate of release increasing with an increased fluoride inclusion, see Examples 2 and 5. By controlling the degree of fluoride inclusion in the particles, they can be tailored to suit the specific needs or requirements of different dental products. The mode of action of the particles in the present invention, in terms of tooth mineralization, is a combination of degradation and corresponding release of bioavailable calcium, magnesium, phosphate, and fluoride ions, and a gradual transformation/crystallization of the particles into fluoride-substituted hydroxyapatite or fluorapatite. Different stages of this crystallization process in a simulated saliva solution are shown in FIG. 4. The core-shell feature of the particles in the present invention is demonstrated by the arrow in FIG. 4A, pointing to the hollow interior of a partly degraded particle shell.

Adding the amorphous calcium magnesium fluoride phosphate particles of the present invention to an oral care product, such as toothpaste, may eliminate or reduce the need of other fluoride salts in product formulations, presenting a competitive advantage and offering a new and innovative option for fluoride treatment to reduce the prevalence of caries or dentin hypersensitivity.

A further aspect of the invention relates to the X-ray amorphous and hollow calcium magnesium fluoride phosphate particles or a composition according to the present invention for use as a medicament. The present invention also relates to the X-ray amorphous and hollow calcium magnesium fluoride phosphate particles or a composition according to the invention for use in prevention or treatment of caries, dentin hypersensitivity or white spot lesions.

‘Treatment’ of caries, dentin hypersensitivity or white spot lesions as used herein does not necessarily mean curative treatment of caries, dentin hypersensitivity or white spot lesions but also encompass inhibition or reduction of the short- and long-term symptoms of the caries, dentin hypersensitivity or white spot lesions. Hence, ‘treatment’ also encompasses delaying onset of the caries, dentin hypersensitivity or white spot lesions, including delaying, preventing onset of symptoms or resolving established pathologies associated with caries, dentin hypersensitivity or white spot lesions, or any other demineralization defect.

Related aspects of the invention define use of the X-ray amorphous and hollow calcium magnesium fluoride phosphate particles or composition according to the invention for the manufacture of a medicament for prevention or treatment of caries, dentin hypersensitivity or white spot lesions.

The present invention also relates to a method of prevention or treatment of caries, dentin hypersensitivity or white spot lesions. The method comprises administering X-ray amorphous and hollow calcium magnesium fluoride phosphate particles or a composition of the invention to a subject to prevent or treat caries, dentin hypersensitivity or white spot lesions.

The subject is a mammalian subject, and preferably a human subject. The particles or composition is administered orally to the subject, preferably in the form of a toothpaste, dentifrice, whitening gel, dental varnish or similar product. Particles according to the present invention can be added as an ingredient in a toothpaste, a dentifrice, a dental varnish, a desensitizing gel, a mineralizing gel, a tooth whitening gel or strip, a dental prophy paste, a mouthwash, a tablet, a chewing gum, a dental sealant, a dental filling material, a dental cement, a dental pulp capping material. An aspect of the invention relates to a dental product selected from the group consisting of a toothpaste, a dentifrice, a dental varnish, a desensitizing gel, a mineralizing gel, a tooth whitening gel or strip, a dental prophy paste, a mouthwash, a tablet, a chewing gum, a dental sealant, a dental filling material, a dental cement, a dental pulp capping material. The dental product comprises a composition according to the invention.

All embodiments disclosed herein relate to all aspects of the present invention and all embodiments may be combined unless stated otherwise.

### EXAMPLES

### Example 1

Particles were synthesized in a conventional jacketed glass reactor. For reference amorphous calcium magnesium phosphate particles without fluoride substitution (ACMP) were prepared, using an aqueous solution with salts and concentrations according to Table 2 with 0 mM NaF. The amorphous calcium magnesium fluoride phosphate (ACMFP) particles were produced by adding NaF at 0.2-5 mM. The phosphate salts and NaF were added to the solution last, as pre-dissolved stock solutions. The pH of the final solution was 7.5. The solution was prepared at room temperature (20° C.), then heated under stirring at a rate of approximately 2° C./min to 80° C. After a holding time at 80-85° C. for 10 min, the precipitated particles were vacuum filtered and washed with deionized water and alcohol. The particles were then allowed to dry in open air containers at room temperature.

TABLE 2
Salt concentrations used in the example

| Salt | Conc. (mM) |
|---|---|
| NaCl | 130 |
| CaCl₂•2H₂O | 1 |
| MgCl₂•6H₂O | 0.6 |
| KH₂PO₄ | 1 |
| Na₂HPO₄•2H₂O | 8 |
| NaF | 0-5 |

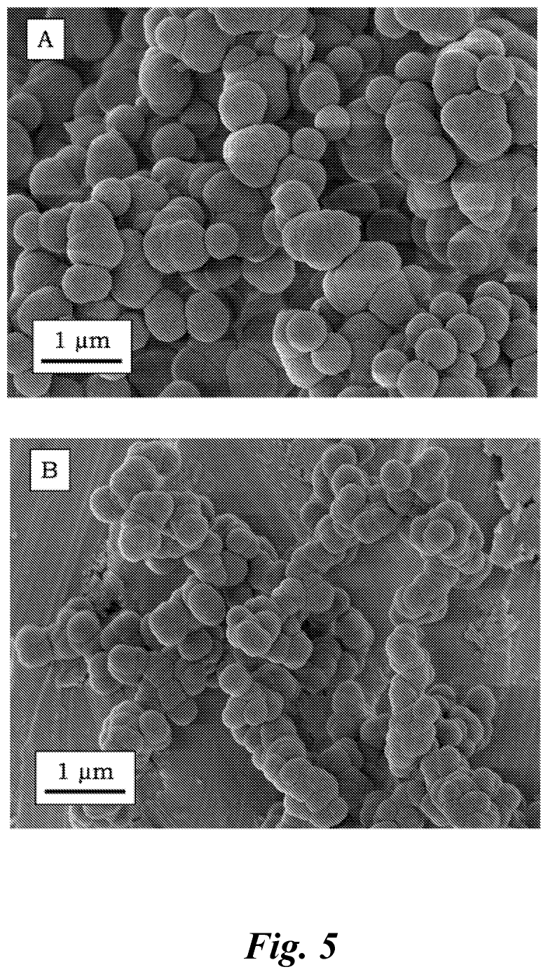

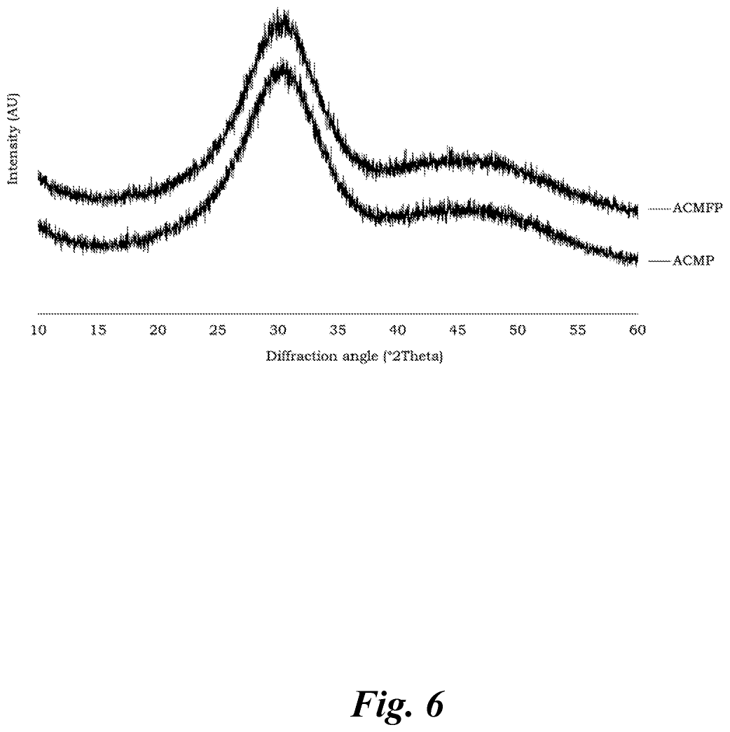

The particles were characterized by scanning electron microscopy (SEM) and X-ray Diffraction (XRD). Appearance of the reference ACMP particles synthesized without fluoride is shown in FIG. 5A, and the appearance of ACMFP particles synthesized with 5 mM NaF is shown in FIG. 5B. The particles were spherical in shape and either present as singular units or as of clusters of fused particles. The general appearance and size of individual particles and clusters were similar for both ACMP and ACMFP particles, demonstrating that the addition of fluoride ions in the synthesis did not significantly alter the particle morphology. Particles synthesized with 0.2 mM NaF and 2 mM NaF had a similar morphology. The composition of the particles in FIG. 5B, synthesized with 5 mM NaF, was quantified by means of energy dispersive X-ray spectroscopy (EDS), and the result is given in Table 3. FIG. 6 shows XRD patterns of the synthesized ACMP (bottom) and ACMFP particles (top), demonstrating that the particles were amorphous.

TABLE 3
Composition (EDS) of particles from the example

| Element | Wt % |
|---|---|
| O | 45.5 |
| Ca | 27.4 |
| P | 14.5 |
| Mg | 4.7 |
| F | 7.9 |

### Example 2

ACMFP particles were synthesized in a conventional jacketed glass reactor by preparing aqueous salt solutions with concentrations and varied fluoride content according to Table 4. The sodium, calcium and magnesium chloride salts were dissolved first, followed by addition of phosphate salts and sodium fluoride as pre-dissolved stock solutions. The pH of the final solution was 7.5. The solution was prepared at room temperature (20° C.), then heated under stirring at a rate of approximately 2° C./min to 80° C. After a holding time at 80-85° C. for 10 min, the precipitated particles were vacuum filtered and washed with deionized water and alcohol. The particles were then allowed to dry in open air containers at room temperature.

TABLE 4
Salt concentrations used in the example

| Salt | Conc. (mM) |
|---|---|
| NaCl | 130 |
| CaCl₂•2H₂O | 1 |
| MgCl₂•6H₂O | 0.6 |
| KH₂PO₄ | 1 |
| Na₂HPO₄•2H₂O | 8 |
| NaF | 0.02-10 |

The obtained particles were spherical in shape and amorphous, similar in appearance and crystallinity as demonstrated in FIG. 5B and FIG. 6.

Fluoride release from particles synthesized with 0.02, 0.2, 2, 5, and 10 mM NaF was measured by adding 0.1% particles by weight to a TISAB II solution under stirring, and measuring the fluoride concentration in solution after 1, 5, 10, and 60 minutes, using a calibrated fluoride ion selective electrode. Results of the fluoride release tests are given in FIG. 7 and clearly demonstrates the increased rate and release of fluoride from particles synthesized with a successively higher fluoride concentration.

The release is characterized by an initial burst, and >90% of the total fluoride measured after 60 minutes was released within the first 10 minutes for all samples. Approximately 80% was released within 5 minutes.

The obtained values were converted to a percentage of the particle mass that was added to solution, yielding the results given in Table 5

### Table 5.

TABLE 5
Amount of fluoride released from the particles

| Synthesis NaF | Particle mass released as |
|---|---|
| concentration (mM) | fluoride within 60 min (%) |
| 0.02 | <0.1% |
| 0.2 | 0.1% |
| 2 | 0.6% |
| 5 | 1.3% |
| 10 | 2.1% |

### Example 3

ACMFP particles were synthesized as described in Example 2, with 2 mM NaF. The obtained particles were spherical in shape and amorphous, similar in appearance and crystallinity as demonstrated in FIG. 5B and FIG. 6.

Composition of the particles according to EDS analysis is given in Table 6.

TABLE 6
Composition (EDS) of particles from the example

| Element | Wt % |
|---|---|
| O | 44.0 |
| Ca | 29.5 |
| P | 14.7 |
| Mg | 4.4 |
| F | 7.4 |

To evaluate the bioactivity of the particles, in terms of their propensity to transform/crystallize into hydroxyapatite or fluorapatite in biological conditions, particles were submersed in a simulated saliva solution composed as described in Table 7 The particles were kept in the solution for 72 h at 37° C., then dried and characterized by SEM and XRD.

TABLE 7
Composition of simulated saliva solution used in the example

| Salt | Conc. (mM) |
|---|---|
| NaCl | 30 |
| KCl | 3 |
| CaCl₂•2H₂O | 1.5 |
| MgCl₂•6H₂O | 0.5 |
| Na₂HPO₄•2H₂O | 2.9 |
| KH₂PO₄ | 2.1 |

Appearance of the particles after 72 h storage in simulated saliva solution at 37° C. is shown in FIG. 8A. The particles had transformed from their original spherical shape into bundles of crystallites with high aspect ratio, i.e., with a needle-like shape. For comparison, particles synthesized without NaF were also stored in the same conditions, and the resulting appearance is shown in FIG. 8B. These particles had also transformed but demonstrated a more flake-like appearance with a lower aspect ratio of the crystals.

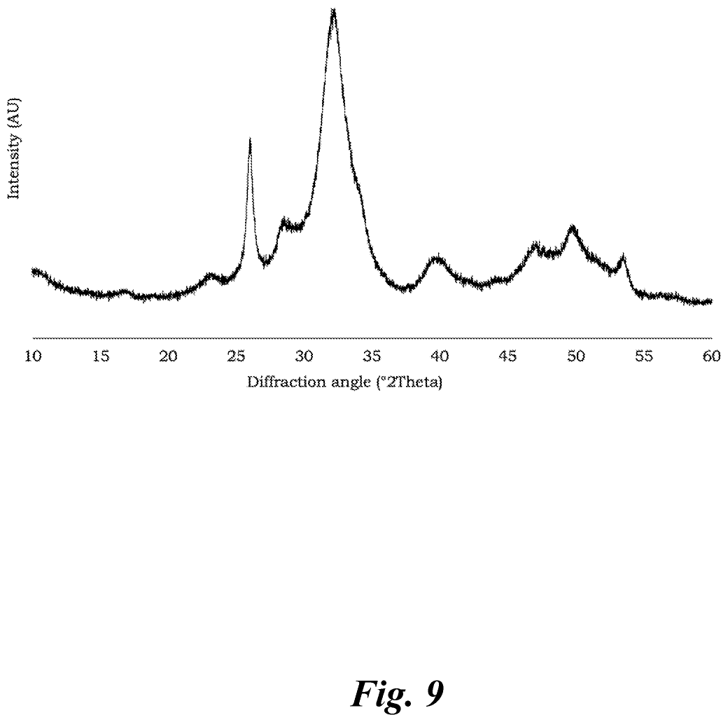

XRD evaluation of the ACMFP particles after storage in simulated saliva demonstrated an increased degree of crystallinity, see FIG. 9. Rietveld refinement of the XRD pattern using Profex v4.3.5 software and quantifying for hydroxyapatite and fluorapatite phases, yielded that the crystalline portion of the particles consisted of approximately 42% hydroxyapatite and 58% fluorapatite.

The synthesized particles were used to perform an in vitro dentin tubule occlusion and mineralization test. The test was performed by sectioning 1 mm thick dentin specimens from extracted human molars, using a low-speed water cooled saw. The dentin specimens were etched using 30% phosphoric acid for 15 seconds to remove the smear layer and expose the dentin tubules, followed by thorough rinsing in deionized water. The dentin specimens were then subjected to a mineralization treatment with formulas containing glycerol and either 5% ACMFP particles or 5% ACMP particles for comparison. A blank treatment formula without any particles was also included for reference. The treatment formulas were applied to dentin specimens twice daily for seven days, using a soft bristled toothbrush and brushing with light hand pressure for 30-45 seconds at each application. In between applications, the dentin specimens were stored in a simulated saliva solution (Table 7) at 37° C., which was exchanged daily. Upon completion of the treatment, one set of specimens were subjected to an acid challenge by submerging them in 2% citric acid (pH≈2) for 30 seconds.

Appearance of dentin surfaces and dentin tubules in cross section after treatment is shown in FIG. 10. The top panel shows the blank reference surface after treatment (A), after treatment and acid challenge (B), and an open dentin tubule in cross section (C). The middle panel shows the dentin surface after treatment with the 5% ACMFP formula (D), after treatment and acid challenge (E), and an example of a completely mineralized dentin tubule deep (>20 μm) beneath the treated surface (F). The bottom panel shows the dentin surface after treatment with the 5% ACMP formula (G), after treatment and acid challenge (H), and an example of a mineralized tubule in cross section (I).

Results of the in vitro test demonstrate that both the ACMFP and ACMP particles are effective in occluding and mineralizing the dentin surface, and that the occlusion and mineralization also occurs deep within the dentin tubules. The cross-section analysis indicates that the newly formed mineral, particularly from the ACMFP particles, is closely adhered to the tubule wall and grows inward to completely seal the tubule. The crystallites were needle-like and similar in appearance to those presented in FIG. 8A, consisting of a combination of hydroxyapatite and fluorapatite, as demonstrated in FIG. 9. The dentine surface treated with the ACMFP formula was also less affected by the acid challenge, as evidenced by FIG. 10E compared to FIG. 10H, which was treated with the ACMP formula. This indicates that the ACMFP particles forms a more acid resistant mineral by crystallizing mostly into fluorapatite, in turn leading to a longer lasting mineralization layer that may offer extended sensitivity relief and enhanced caries protection compared to the ACMP particles.

### Example 4

Amorphous calcium magnesium fluoride phosphate particles according to the present invention were synthesized in a two-solution, continuous flow mixing process. In the process, two separate salt solutions were prepared; one containing 55 mM CaCl₂ and 33 mM MgCl₂; and one containing 220 mM Na₂HPO₄ and 10 mM NaF. The solutions were prepared by dissolving the salts in deionized water at 25° C. The solutions were then heated separately to 85° C. and pumped at a similar flow rate to a Y-shaped mixing point, where the solutions react to form particles of the present invention. The formed particles were collected by filtration and washed with deionized water to remove residual salts and counter ions.

The retrieved particle slurry, consisting of approximately 20 wt % particles and 80 wt % water, was mixed with glycerol, and the mixture was dried in a forced convection oven at 80° C. to remove the excess water. The dried product was then homogenized to obtain a smooth and stable paste consisting of approximately 35 wt % particles, 60 wt % glycerol, and 5 wt % residual water. The apparent viscosity of the paste was approximately 5 000 mPa-s, as measured using a rotational viscometer at 20° C.

The produced particles were spherical in shape and ranging from approximately 100 to 400 nm in diameter, appearing as those demonstrated in FIG. 2. Particle size distribution was further measured using dynamic light scattering (DLS) after dispersion by ultrasound in ethanol. A single peak at 238±96 nm was recorded, and the Z-average particle size was 300 nm. The particles were amorphous, with an XRD pattern as that shown in FIG. 3. Compositional analysis using inductively coupled plasma-optical emission spectroscopy (ICP-OES) yielded that the particles contained 19 wt % calcium, 6 wt % magnesium, and 18 wt % phosphorous (corresponding to 55 wt % phosphate). It can be assumed that the remaining compounds are mostly water and fluoride since nothing else has been added to the particles. Fluoride, hydrogen and oxygen are not detectable in ICP-OES.

The BET surface area of the particles, as measured using N₂ adsorption, was 26 m²/g.

The synthesis starting concentration of NaF used in the current example was 10 mM, corresponding to 190 ppm F⁻. The level of fluoride in the filtrate was measured to 60 ppm F⁻, using a fluoride ion selective electrode, indicating that close to 70% of the available fluoride was consumed in the reaction and could therefore be incorporated into the particles. Based on this, the theoretical maximum fluoride content in the particles of this example was 1.3 wt %.

The fluoride ion release profile from the particles was determined by adding 1 mg/mL of the particle/glycerol paste to a 1% TISAB II solution under stirring, and measuring fluoride concentration at start and after 1, 5, 10, 20, 30, and 60 minutes. The fluoride release curve is shown in FIG. 11, demonstrating that most of the fluoride was released during the initial 10 minutes. The result indicates that approximately 1 wt % of the particle mass added to the TISAB II solution was released as fluoride during the 60 minutes of measurement, corresponding to 82% of the theoretical maximum fluoride content in the particles.

The paste consisting of approximately 35 wt % particles, 60 wt % glycerol, and 5 wt % residual water was placed in a sealed container and subjected to accelerated storage at 40° C. for a period of three months. Subsequent SEM and XRD analysis demonstrated that the particles had remained stable in the paste, preserving their spherical shape and overall morphology as well as their amorphous character, see FIGS. 16a and 16b.

### Example 5

Amorphous calcium magnesium fluoride phosphate particles according to the present invention were synthesized with varied fluoride content in a two-solution continuous flow mixing process. The contents and concentrations of the two starting solutions are given in Table 8. The two solutions were heated separately to 80° C., then pumped at an equal flow rate using a dual head peristaltic pump to a converging mixing point where immediate precipitation occurred. The formed particles were collected by vacuum filtration and washed with deionized water, followed by drying at 110° to form a fine powder.

TABLE 8
Salt concentrations used in the example

| Solution #1 | Molar amt. (mM) |
|---|---|
| CaCl₂•2H₂O | 55 |
| MgCl₂•6H₂O | 33 |
| Solution #2 | Molar amt. (mM) |
| Na₂HPO₄•2H₂O | 220 |
| NaF | 0-40 |

The particles were analyzed in SEM coupled with an energy dispersive x-ray spectroscopy detector (EDS), providing both high resolution images and compositional information about the particles. In the compositional EDS analysis, the elements known to be present and therefore quantified were O, Ca, P, Mg, and F, and the total was tallied to 100% for comparison between samples. Crystallinity of the particles was analyzed by XRD. Fluoride release from the particles was analyzed by adding 50 mg of particles to 100 mL of TISAB II solution, resulting in a 0.5 mg/mL (500 ppm) particle concentration. The solutions were continuously stirred and the fluoride concentration was measured after 1, 5, 10, and 30 minutes using a calibrated fluoride ion selective electrode.

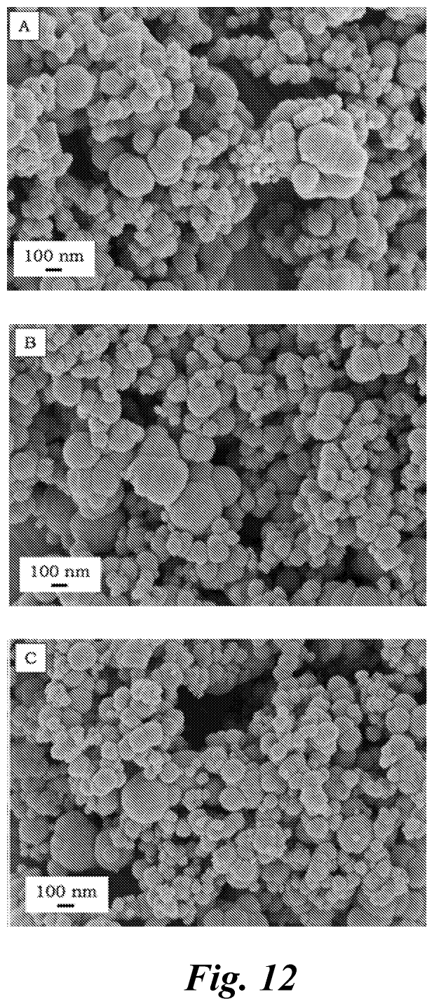

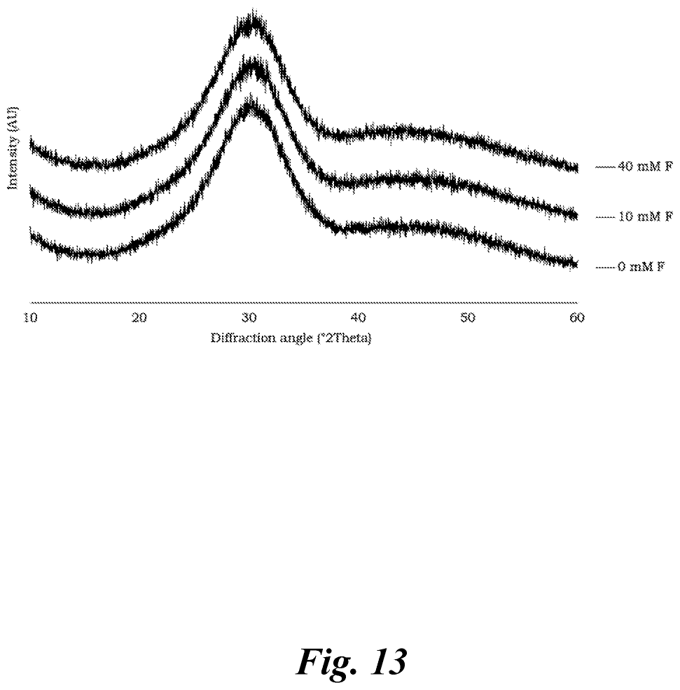

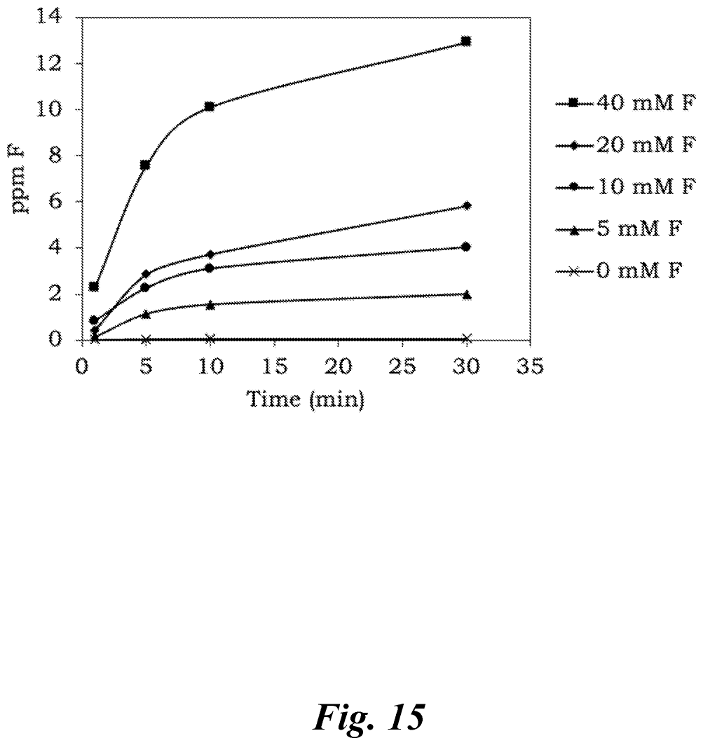

Appearance of particles in SEM are shown in FIG. 12, displaying particles synthesized with 0 mM NaF in (A), 10 mM NaF in (B), and 40 mM NaF in (C). The individual spherical particles ranged in size from approximately 50 to 400 nm in diameter. Particles synthesized with 5 mM NaF and 20 mM NaF had a similar appearance. The XRD patterns of particles synthesized with 0, 10, and 40 mM NaF are shown in FIG. 13, demonstrating an amorphous character in all instances. FIG. 14 and Table 9 shows the composition of the particles, as determined by EDS. It demonstrates the increased content of fluoride in the particles in line with an increased synthesis concentration of NaF, going from 0 wt % F in particles synthesized without NaF, to almost 6 wt % F in particles synthesized with 40 mM NaF. The fluoride release from the particles is shown in FIG. 15, demonstrating the successively increased rate and total release from particles synthesized with an increased fluoride concentration. Considering the 500 ppm particle concentration in the measurement solution, the fluoride release from the particles synthesized with 40 mM NaF corresponds to approximately 2.6 wt % of the added particles released within 30 minutes.

TABLE 9
Composition (EDS) of particles according to synthesis concentration
of NaF

| | 0 mM | 5 mM | 10 mM | 20 mM | 40 mM NaF |
|---|---|---|---|---|---|
| Element | NaF | NaF | NaF | NaF | NaF |
| O | 54.2% | 48.4% | 50.1% | 50.3% | 48.6% |
| Ca | 23.2% | 25.5% | 23.0% | 24.7% | 25.7% |
| P | 14.6% | 19.0% | 17.9% | 15.5% | 12.8% |
| Mg | 7.9% | 5.9% | 6.4% | 6.3% | 7.1% |
| F | 0.1% | 1.2% | 2.6% | 3.2% | 5.8% |

1. X-ray amorphous and hollow calcium magnesium fluoride phosphate particles having a mean diameter ranging from 50 to 500 nm, wherein the X-ray amorphous and hollow calcium magnesium fluoride phosphate particles comprise a respective shell, wherein the respective shell comprises:
15-30 wt % calcium of the total weight of the X-ray amorphous and hollow calcium magnesium fluoride phosphate particles;
50-65 wt % phosphate of the total weight of the X-ray amorphous and hollow calcium magnesium fluoride phosphate particles;
4-8 wt % magnesium of the total weight of the X-ray amorphous and hollow calcium magnesium fluoride phosphate particles;
1-10 wt % fluoride of the total weight of the X-ray amorphous and hollow calcium magnesium fluoride phosphate particles; and
wherein the balance is water.

2. The particles according to claim 1, wherein the respective shell comprises:
18-30 wt % calcium, preferably 20-25 wt % calcium, of the total weight of the X-ray amorphous and hollow calcium magnesium fluoride phosphate particles;
50-60 wt % phosphate, preferably 52-57 wt % phosphate, of the total weight of the X-ray amorphous and hollow calcium magnesium fluoride phosphate particles;
5-8 wt % magnesium, preferably 6-7 wt % magnesium, of the total weight of the X-ray amorphous and hollow calcium magnesium fluoride phosphate particles;
3-10 wt % fluoride, preferably 3.2-5.8 wt % fluoride, of the total weight of the X-ray amorphous and hollow calcium magnesium fluoride phosphate particles; and
wherein the balance is water.

3. The particles according to claim 2, wherein the respective shell comprises:
20-25 wt % calcium of the total weight of the X-ray amorphous and hollow calcium magnesium fluoride phosphate particles;
52-57 wt % phosphate of the total weight of the X-ray amorphous and hollow calcium magnesium fluoride phosphate particles;
6-7 wt % magnesium of the total weight of the X-ray amorphous and hollow calcium magnesium fluoride phosphate particles;
3.2-5.8 wt % fluoride of the total weight of the X-ray amorphous and hollow calcium magnesium fluoride phosphate particles; and
wherein the balance is water.

4. The particles according to claim 1, wherein the respective shell comprises 3.2-10 wt % fluoride.

5. The particles according to claim 1, wherein:
the amount of calcium, magnesium and phosphorus in wt % is determined using inductively coupled plasma-optical emission spectroscopy (ICP-OES);
the phosphorous (P) is presumed to be present as phosphate (PO₄); and
the amount of fluoride is measured using energy-dispersive X-ray spectroscopy (EDS) or a fluoride ion selective electrode.

6. The particles according to claim 1, wherein the X-ray amorphous and hollow calcium magnesium fluoride phosphate particles are substantially spherical.

7. The particles according to claim 1, wherein the water is bound water.

8. A composition comprising a paste-forming compound and the X-ray amorphous and hollow calcium magnesium fluoride phosphate particles according to claim 1.

9. The composition according to claim 8, wherein the paste-forming compound is selected from the group consisting of glycerol, triglyceride, polyethylene glycol, propylene glycol, polypropylene glycol, polyvinyl alcohol, mineral oil, liquid paraffin, and any mixture thereof.

10. The composition according to claim 9, wherein
the paste-forming compound is glycerol; and
the composition comprises 20-50 wt % of the X-ray amorphous and hollow calcium magnesium fluoride phosphate particles, based on the total weight of the composition.

11. A method of manufacturing X-ray amorphous and hollow calcium magnesium fluoride phosphate particles according to claim 1, wherein the method comprises the steps of:
formation of:
a first salt solution comprising 10-200 mM Ca²⁺ and 5-120 mM Mg²⁺; and
a second salt solution comprising 0-100 mM H₂PO₄⁻, 30-300 mM HPO₄²⁻, and 1-150 mM F⁻,
heating the first respectively the second salt solution separately to 60-90° C., forming a first, respectively second heated solution;
mixing the first heated solution with the second heated solution in a 1:1 volume ratio forming a mixed solution;
precipitation of particles from the mixed solution obtaining a slurry comprising particles; and
retrieving of the particles from the slurry.

12. The method according to claim 11, further comprising washing of the particles retrieved from the slurry.

13. A medicament comprising the X-ray amorphous and hollow calcium magnesium fluoride phosphate particles according to claim 1.

14. A method to prevent or treat caries comprising using the X-ray amorphous and hollow calcium magnesium fluoride phosphate particles according to claim 1.

15. A method to prevent or treat dentin hypersensitivity comprising using the X-ray amorphous and hollow calcium magnesium fluoride phosphate particles according to claim 1.

16. A method to prevent or treat white spot lesions comprising using the X-ray amorphous and hollow calcium magnesium fluoride phosphate particles according to claim 1.

17. A dental product comprising a composition according to claim 8, wherein the dental product is selected from the group consisting of a toothpaste, a dentifrice, a dental varnish, a desensitizing gel, a mineralizing gel, a tooth whitening gel or strip, a dental prophy paste, a mouthwash, a tablet, a chewing gum, a dental sealant, a dental filling material, a dental cement, a dental pulp capping material.

18. A medicament comprising the composition according to claim 8.

19. A method to prevent or treat caries, dentin hypersensitivity, or white spot lesions comprising using the composition according to claim 8.
